(19) United States
(12) Patent Application Publication
(10) Pub. No.: US 2023/0248248 A1
(43) Pub. Date: Aug. 10, 2023

(54) PATIENT TEMPERATURE MONITORING SYSTEM

(71) Applicant: American Sterilizer Company, Mentor, OH (US)

(72) Inventors: James A. Petrucci, Kirtland, OH (US); David Jesurun, South Euclid, OH (US); David A. Westenfelder, II, Mantua, OH (US); Joseph J. Groszek, Rocky River, OH (US)

(21) Appl. No.: 18/167,192

(22) Filed: Feb. 10, 2023

Related U.S. Application Data
(60) Provisional application No. 63/308,966, filed on Feb. 10, 2022.

(51) Int. Cl. A61B 5/01; A61B 90/35; A61B 5/00

(52) U.S. Cl. CPC A61B 5/015; A61B 90/35; A61B 5/6887; A61B 5/0077; A61B 2562/0271; A61B 2505/05

(57) ABSTRACT

A surgical lighting system includes a surgical lighthead including a lighthead housing including one or more light emitting elements therein that are arranged to emit light toward a region of interest. In some embodiments a temperature sensor is mounted to the lighthead housing and arranged such that it measures the temperature of a location in the region of interest. In other embodiments, a temperature sensor is external to the lighthead and configured to measure the temperature of a location in the region of interest. A controller is operatively coupled to the temperature sensor and configured to control temperature at the region of interest based at least in part on the temperature measured by the temperature sensor.

### FIELD OF THE INVENTION

The technology of the present disclosure relates generally to a patient temperature monitoring system, and more specifically to a surgical lighting system, medical device support system and/or suspension system including a temperature sensor for monitoring the temperature at the surgical site of a patient.

### BACKGROUND

A patient's core body temperature is typically monitored during a medical procedure. For example, it is generally recommended that body temperature should be measured in a patient having general anesthesia exceeding 30 minutes in duration. Sites for continuous core temperature monitoring include the esophagus, nasopharynx, tympanic membrane, bladder, and the pulmonary artery. Monitoring core body temperature may be used in the control of active warming and/or cooling systems in order to prevent hypothermia or hyperthermia.

Control of the localized temperature at the surgical site can also be of importance, especially when a delicate organ is exposed at the surgical site. However, the core body temperature of a patient may not accurately reflect the localized temperature at the surgical site. This is because the surgical site may be exposed to environmental conditions that cause localized temperature fluctuations at the surgical site that are not readily detected or controllable using core body temperature. For example, exposure of the surgical site to the room temperature may cause the localized temperature of the surgical site to be lower than the core body temperature of the patient. As another example, lightheads for medical device support systems, suspension systems, and/or other carry systems are used in health treatment settings such as hospital examination rooms, clinics, surgery rooms and emergency rooms to illuminate a region of interest (e.g., surgical treatment site or other medical site) below or proximate the lighthead. Because surgical lights direct thermal energy to their focal point, this may cause the temperature of the surgical site to be higher than the core body temperature of the patient. Other equipment in the vicinity of the surgical site may also affect the room temperature in the vicinity of the surgical site, which may in-turn affect the temperature of the surgical site. Common examples of equipment that can affect the environmental conditions are electro surgery tools, surgical energy devices, surgical tables, camera system, light source, bypass machine, patient warmer, insufflator, EKG, anesthesia cart, and the like.

The impact that the environmental conditions have on the localized temperature of the surgical site can also change over time throughout the medical procedure. For example, in an operating room, equipment concentrated around the surgical table can be used or repositioned at different times during the medical procedure. Healthcare professionals and staff also typically gather around the surgical table and can change position over time. These factors and changes can all have an impact on the localized temperature at the surgical site.

Accordingly, there remains a need for further contributions in this area of technology.

### SUMMARY OF INVENTION

The present disclosure relates to a patient temperature monitoring system that measures the temperature in a region of interest, such as the surgical site of a patient. In some embodiments, the patient temperature monitoring system is included as part of a surgical lighting system, medical device support system, suspension system, and/or carry system. An exemplary application includes the patient temperature monitoring system integrated with a surgical light such as those used in operating rooms to provide light to a specific area of the room (e.g., the region of interest). In some embodiments, the temperature sensor is arranged in the handle of the surgical lighthead. Other exemplary applications include the patient temperature monitoring system mounted to a dedicated suspension arm or boom. In other embodiments, the patient temperature monitoring system is affixed to a wall or ceiling of the room.

Embodiments of the present disclosure allow for monitoring and control of the temperature at the region of interest, such as the surgical site of a patient. This can allow for the control system and/or surgical staff to more quickly make decisions to warm or cool the patient. Exemplary control can include one or more of the control of light intensity and/or light output distribution from the lighthead, the control of one or more devices of an active warming/cooling system, and the control of one or more environmental temperature control systems. Examples include control of one or more warming or cooling peripheral devices of a patient warming/cooling system (e.g., blanket, underbody pad, etc.), controlling the intensity of the one or more light emitting elements of the lighthead, control of room temperature, and the like.

In accordance with one aspect of the present disclosure, a system includes: a surgical lighthead including a lighthead housing including one or more light emitting elements therein that are arranged to emit light toward a region of interest; a temperature sensor mounted to the lighthead housing and arranged such that it measures the temperature of a location in the region of interest; and a controller operatively coupled to the temperature sensor and configured to control temperature at the region of interest based at least in part on the temperature measured by the temperature sensor.

In some embodiments, the temperature sensor is an infrared thermometer.

In some embodiments, the temperature sensor is an infrared camera.

In some embodiments, the system further includes a handle mounted to the lighthead housing, wherein the temperature sensor is within the handle.

In some embodiments, the region of interest is a surgical site.

In some embodiments, the controller is configured to control environmental temperature based at least in part on the temperature measured by the temperature sensor.

In some embodiments, the controller is configured to control an HVAC system based at least in part on the temperature measured by the temperature sensor.

In some embodiments, the controller is configured to control a warming blanket based at least in part on the temperature measured by the temperature sensor.

In some embodiments, the controller is configured to control a heated underbody pad based at least in part on the temperature measured by the temperature sensor.

In some embodiments, the controller is configured to control a heated headrest based at least in part on the temperature measured by the temperature sensor.

In some embodiments, the controller is configured to control intensity of the one or more light emitting elements based at least in part on the temperature measured by the temperature sensor.

In some embodiments, the controller is configured to control light output distribution of the one or more light emitting elements based at least in part on the temperature measured by the temperature sensor.

In accordance with another aspect of the present disclosure, a surgical lighting system includes: a lighthead housing including one or more light emitting elements therein that are arranged to emit light toward to a region of interest; a handle mounted to the lighthead housing, the handle including a handle housing having a sufficient size to be gripped by the human hand; and a temperature sensor mounted within the handle housing and arranged such that it measures the temperature of a location in the region of interest.

In some embodiments, the temperature sensor is an infrared thermometer.

In some embodiments, the temperature sensor is an infrared camera.

In accordance with another aspect of the present disclosure, a system includes: a medical device suspension system; a surgical lighthead mounted to the medical device suspension system and comprising a lighthead housing including one or more light emitting elements therein that are arranged to emit light toward to a region of interest; a temperature sensor external to the lighthead and configured to measure the temperature of a location in the region of interest; and a controller operatively coupled to the temperature sensor and configured to control temperature at the region of interest based at least in part on the temperature measured by the temperature sensor.

In some embodiments, the temperature sensor is an infrared thermometer.

In some embodiments, the temperature sensor is an infrared camera.

In some embodiments, the temperature sensor is mounted to the medical device suspension system.

In some embodiments, the temperature sensor is not mounted to the medical device suspension system.

In some embodiments, the region of interest is a surgical site.

In some embodiments, the controller is configured to control room temperature based at least in part on the temperature measured by the temperature sensor.

In some embodiments, the controller is configured to control a patient warming system based at least in part on the temperature measured by the temperature sensor.

In some embodiments, the controller is configured to control a warming blanket based at least in part on the temperature measured by the temperature sensor.

In some embodiments, the controller is configured to control a heated underbody pad based at least in part on the temperature measured by the temperature sensor.

In some embodiments, the controller is configured to control a heated headrest based at least in part on the temperature measured by the temperature sensor.

In some embodiments, the controller is configured to control intensity of the one or more light emitting elements based at least in part on the temperature measured by the temperature sensor.

In some embodiments, the controller is configured to control light output distribution of the one or more light emitting elements based at least in part on the temperature measured by the temperature sensor.

These and further features will be apparent with reference to the following description and attached drawings which set forth certain illustrative embodiments of the invention. These embodiments are indicative, however, of but a few of the various ways in which the principles of the invention may be employed. Other objects, advantages, and novel features according to aspects of the invention will become apparent from the following detailed description when considered in conjunction with the drawings. The invention includes all changes, modifications and equivalents coming within the spirit and terms of the claims appended hereto.

Features that are described and/or illustrated with respect to one embodiment may be used in the same way or in a similar way in one or more other embodiments and/or in combination with or instead of the features of the other embodiments.

### BRIEF DESCRIPTION OF THE DRAWINGS

The annexed drawings, which are not necessarily to scale, show various aspects of the present disclosure.

FIG. 1 is a side elevation view of an overall configuration of a medical device support system in accordance with an embodiment of the present disclosure, showing a top of a left positioned lighthead and a bottom of a right positioned lighthead.

FIG. 2 is a side cross section view of a lighthead in accordance with an embodiment of the present disclosure, showing a housing base, a housing cover, and internal components of the lighthead and handle.

FIG. 3 is a perspective side view of a handle in accordance with an embodiment of the present disclosure having a handle housing including a grip portion.

FIG. 4 is a schematic side view of an exemplary room including a medical device support system.

FIG. 5 is a perspective view of a lighthead in accordance with an embodiment of the present disclosure.

FIG. 6 is a schematic side view of an exemplary medical device support system arranged relative to an operating table.

FIGS. 7A-7D show respective embodiments of field of view relative to a region of interest illuminated by a lighthead.

FIG. 8 is a perspective view of a lighthead in accordance with another embodiment of the present disclosure.

FIG. 9 is a schematic side view of an exemplary medical device support system arranged relative to an operating table.

FIG. 10 is a side elevation view of an overall configuration of a medical device support system in accordance with another embodiment of the present disclosure, showing a top of a left positioned lighthead and a suspension arm including a temperature sensor.

FIG. 11 is a schematic side view of an exemplary medical device support system arranged relative to an operating table.

FIG. 12 is a schematic side view of an exemplary medical device support system arranged relative to an operating table.

FIG. 13 is a schematic block diagram of an exemplary control system.

FIGS. 14 and 15 are schematic block diagrams of exemplary temperature control systems.

FIG. 16 is a flow chart showing an exemplary process for determining a measured temperature in the region of interest.

FIG. 17 is a flowchart showing an exemplary process for controlling temperature at the region of interest based at least in part on the temperature measured by the sensor.

### DETAILED DESCRIPTION

For the purpose of promoting an understanding of the principles of the present disclosure, reference will now be made to the embodiments illustrated in the drawings and specific language will be used to describe the same. It will nevertheless be understood that no limitation of the scope of the present disclosure is thereby intended. The figures are not necessarily to scale. Features that are described and/or illustrated with respect to one embodiment may be used in the same way or in a similar way in one or more other embodiments and/or in combination with or instead of the features of the other embodiments. Any alterations and further modifications of the described embodiments, and any further applications of the principles of the present disclosure as described herein, are contemplated as would normally occur to one skilled in the art to which the present disclosure relates.

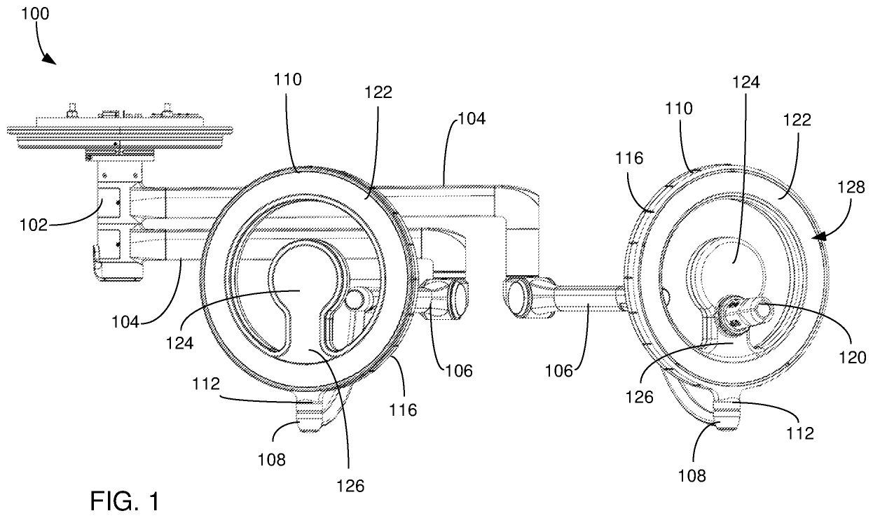

With reference to FIG. 1, an exemplary medical device support system is shown at 100. The medical device support system 100 includes a central shaft or support column 102 that is suspended from the ceiling, and two generally horizontal extension arms 104 mounted to the shaft 102 for rotational movement about the central shaft 102. In other implementations, the central shaft 102 could be mounted to a wall or stand rather than the ceiling. Two load balancing arms 106 are pivotably mounted to the distal ends of the respective extension arms 104. Yoke assemblies 108 are mounted to the distal ends of the respective load balancing arms 106. The yoke assemblies 108, in turn, support respective lightheads 110 for multi-axis movement relative to the load balancing arms 106. Each lighthead 110 includes a bushing or other coupling member 112 that rotatably connects the lighthead 110 to the distal end of an arm of a respective yoke assembly 108, as shown. The load balancing arms 106 and yoke assemblies 108 enable positioning of the lightheads 110 to a desired orientation relative to, for example, a patient operating table and healthcare professionals in the operating room.

The exemplary medical device support system shown in FIG. 1 includes two lightheads 110, each mounted to a respective extension arm 104, load balancing arm 106, and yoke assembly 108. It will be appreciated that in other embodiments, the medical device support system may include more or fewer lightheads. It will also be appreciated that the medical device support system may include other accessories mounted to the central shaft 102.

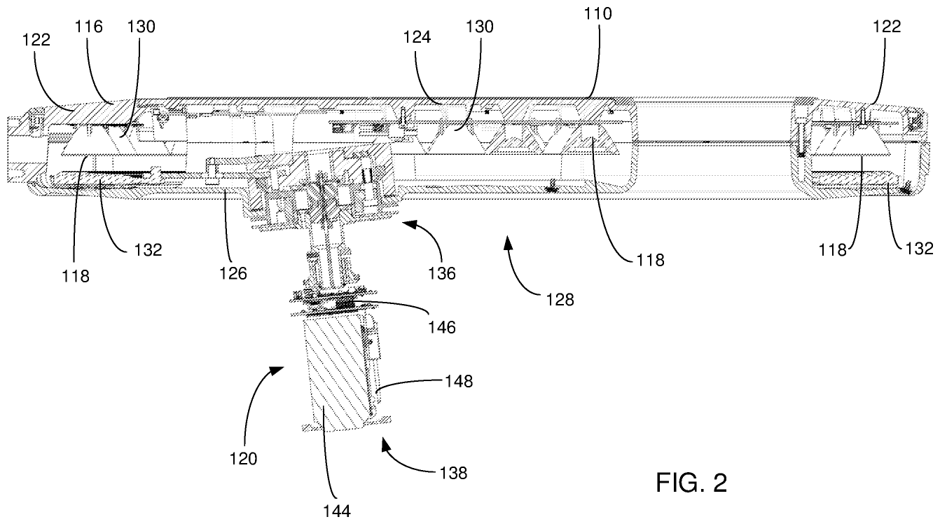

With additional reference to FIG. 2, the lighthead 110 includes a housing 116, one or more light emitting elements 118 mounted inside the housing, and a handle 120 mounted to the housing to enable a healthcare professional or other individual to adjust the position of the lighthead according to the needs of a specific medical procedure. In the illustrated example, the lighthead 110 includes an annular shape outer portion 122, an inner round portion 124 that is concentric with the outer portion, and a radially protruding arm 126 that connects the annular shape outer portion 122 to the inner round portion 124. The handle is connected to the radially protruding arm.

As shown in FIG. 2, the housing 116 supports the light emitting elements 118. The light emitting elements 118 may in some embodiments include one or more solid-state light emitters. Exemplary solid-state light emitters include such devices as light emitting diodes (LEDs), laser diodes, and organic LEDs (OLEDs). The LEDs may be broad spectrum LEDs (e.g., white light emitters) or LEDs that emit light of a desired color or spectrum (e.g., red light, green light, blue light, or ultraviolet light). In other embodiments, the LEDs may be a mixture of broad-spectrum LEDs and LEDs that emit narrow-band light of a desired color, or a mixture of LEDs that emit light of different respective colors or spectrum. In some embodiments, the solid-state light emitters constituting the light emitting elements 118 all generate light having the same nominal spectrum. In other embodiments, at least some of the solid-state light emitters constituting the light emitting elements 118 generate light that differs in spectrum from the light generated by the remaining solid-state light emitters. In other embodiments, the light emitting elements 118 may include one or more other types of light sources. Non-limiting examples of other types of light sources include incandescent and gas discharge light sources. In still other embodiments, the light emitting elements 118 may include a combination of solid-state light emitters and one or more of the above other types of light sources.

Light from the light emitting elements is emitted from a light emitting face 128 (i.e., light emitting side) of the lighthead. The lighthead may include one or more elements configured to affect the light output distribution of the light from the lighthead. With continued reference to FIG. 2, collimators 130 are mounted to the inside surface of the housing 116 and in the light emitting paths of the respective light emitting elements 118. Each collimator may be associated with a respective light emitting element 118 and may be arranged to collect and direct, and/or collimate, the light emitted from the associated light emitting element 118 into a narrowed beam. In the illustrative embodiment, the housing 116 also includes a lens 132, that is shaped to redirect light emitted from the light emitting elements and passing therethrough. The lens 132 can take on any form for spreading and/or bending the light emitted by the light emitting elements 118. The lens may be adjusted to adjust the spreading, focusing, and and/or bending the light. In other embodiments, the lens and/or the collimators may be omitted from the lighthead.

A controller (402, 604) controls the light emitting elements 118 to emit light to a region of interest (e.g., surgical treatment site or other medical site) below or proximate the lighthead 110. For example, a controller may control the light sources 118 of the annular shape outer portion 122 and the inner portion 124 to emit light to a region of interest below the lighthead 110. Control of the respective light sources 118 may be performed, for example, collectively, individually, in groups, by section, or in any other suitable manner. In some embodiments, the controller may be provided as part of the lighthead 110. In other embodiments, the controller may be implemented elsewhere in the medical device support system 100, for example external to the lighthead 110, or the controller may be implemented external to the medical device support system 100. In yet other embodiments, the controller may be partitioned into multiple, separated locations (i.e. in the lighthead 110, elsewhere in the medical device support system 100, and external to the medical device support system).

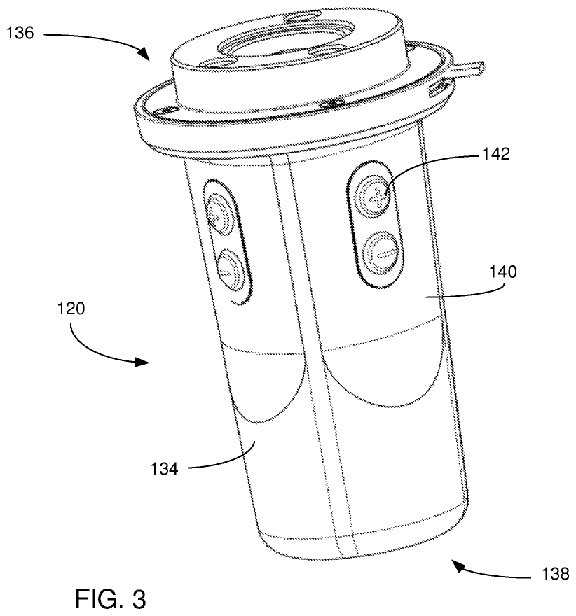

The handle 120 is attached to the light head housing and extends between a proximate end proximate the housing and a distal end distal the housing. With reference to FIG. 3, the handle 120 includes a handle housing 134 that has generally tubular shape extending between the proximate end 136 and distal end 138. The tubular shape may be cylindrical in shape, as shown, or non-cylindrical in shape. The handle 120 includes a grip portion 140 including one or more buttons 142. In some embodiments, the one or more buttons provide a user interface for controlling one or more attributes of the emitted light from the lighthead 110. For example, the one or more buttons may be used as an input for a user to adjust the color temperature, intensity, and/or distribution of the light. The handle housing 134, including the grip portion 140 thereof, has a sufficient size to be gripped by a human hand meaning that the outermost diameter or perimeter of the handle housing 134 is selected to enable a human hand to be wrapped around the handle housing.

It will be appreciated that the specific configuration of the lighthead 110, including the shape of the housing, the arrangement of the light emitting elements 118, and the location and configuration of the handle 120 can be provided in any suitable configuration. For example, an annular shape outer portion 122 and the inner round portion 124 need not be in concentric relation to one another and instead can be arranged by the protruding arm in eccentric relation to one another. In another example, the inner round portion 124 of the lighthead 110 may be omitted; and in such form, only the annular shape outer portion 122 emits light to the region of interest (e.g., surgical treatment site or other medical site) below or proximate the lighthead. In another example, the housing may be configured as a different shape (e.g., rectangle, square, circle, hexagon, octagon, etc.) and the light emitting elements can be accordingly arranged in the housing. In another example, the handle can be provided at a different location on the light emitting side of the housing, on a perimeter of the housing, or on the side of the housing opposite the light emitting side.

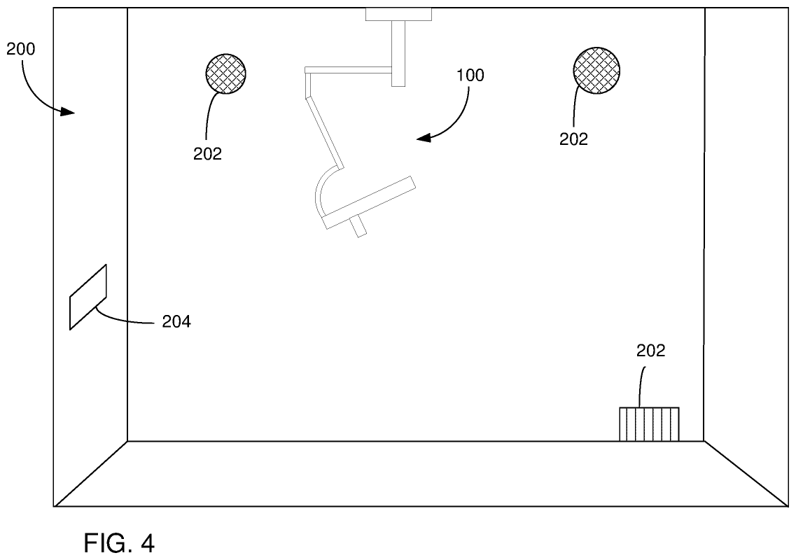

With reference to FIG. 4, the medical device support system 100 may be installed in a room 200 to provide light to a specific area of the room. The room may be, for example, an operating room. In the embodiment shown, the medical device support system 100 is fixed to the ceiling. One or more environmental temperature control systems may be associated with the room 200. For example, an HVAC system may provide heating and/or cooling in the room. The HVAC system may include components such as a blower motor, filter, fan, etc. One or more air ducts, vents, registers, and/or returns 202 included as part of the HVAC system may be present in the room at predetermined locations to effect heating and/or cooling. One or more user inputs 204 such as a control panel may be included that allow for a user to adjust the environmental conditions in the room (e.g., room temperature).

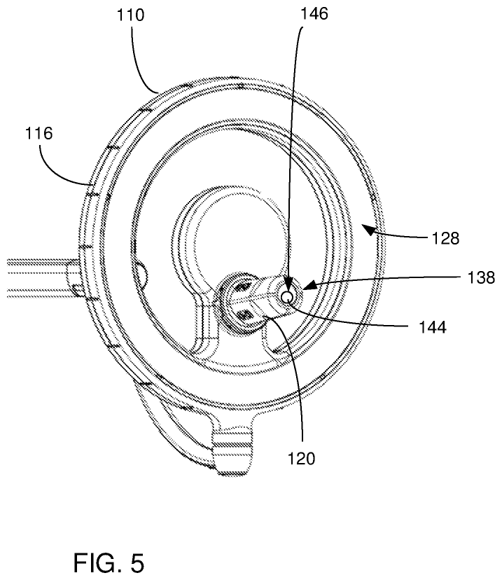

In some embodiments, the lighthead includes a temperature sensor 144 (FIG. 5). The temperature sensor is arranged such that it measures the temperature of a location in the region of interest that is illuminated by the one or more light emitting elements. Integration of the temperature sensor into the lighthead allows for placement of the sensor a location not likely/often obstructed by the operating room staff. Furthermore, the temperature sensor will be aimed when a surgical light is aimed at the surgical site.

With reference to FIG. 5, in some embodiments, the temperature sensor 144 is housed within the handle of the lighthead. The temperature sensor 144 may be aligned to measure the temperature of the location in the region of interest from the distal end 138 of the handle. As an example, the distal end of the handle may include a port 146 through which the sensor is aimed. In another example, the distal end of the handle may include an optically translucent cover through which the sensor measures the temperature of the location.

In some embodiments, the temperature sensor 144 is embodied as an infrared temperature sensor. In some examples, the infrared temperature sensor is an infrared thermometer that reacts to infrared radiation emitted by the object being measured. A lens may focus the infrared thermal radiation from the object on the sensor. The sensor converts the radiant power to an electrical signal. In other examples, the temperature sensor is embodied as an infrared camera that creates an image of the spatial distribution of infrared radiation. Images and/or video of a field of view can be captured by the camera, which may include a target in a region of interest illuminated by the one or more light emitting elements. In other examples, one or more other types of temperature sensors can be used. For example, in some embodiments the temperature sensor is embodied as an ultrasonic temperature sensor.

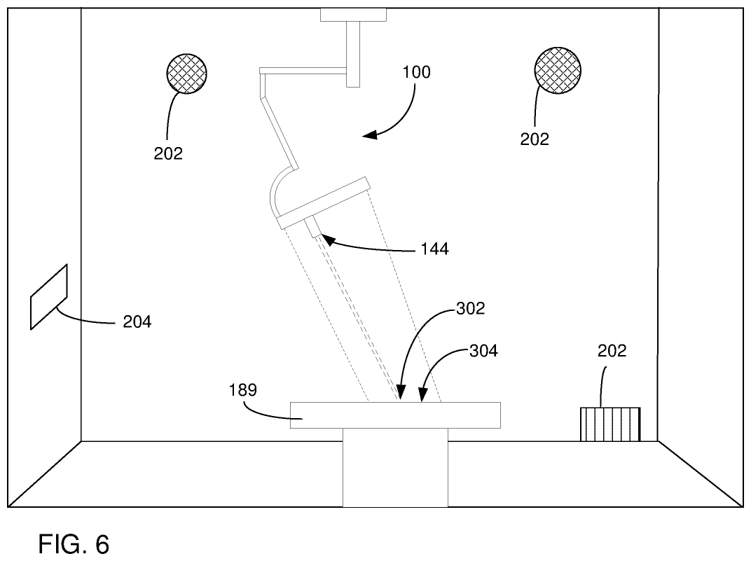

FIG. 6 shows a schematic side view of an exemplary medical device support system 100 arranged relative to an operating table. The lighthead may be arranged such that it is a predetermined distance from the region of interest. Adjustment of the lighthead relative to the region of interest may be performed using the extension arm 104, load balancing arm 106, and/or yoke assembly 108. In an example, the lighthead may be adjusted such that it is a distance of about one meter from the region of interest. The area illuminated by the one or more light emitting elements 118 is the region of interest. The region of interest 304 may be formed by the light emitting elements 118 that emit light and collimators and/or lenses that aim, redirect, spread, converge, and or focus the light. The temperature sensor measures the temperature of a location relative to the region of interest. The region of interest 304 may include a specific target, such as a surgical site of a patient on a surgical table 189, and the location measured or imaged by the temperature sensor may coincide with the region of interest or may be a subset of the region of interest.

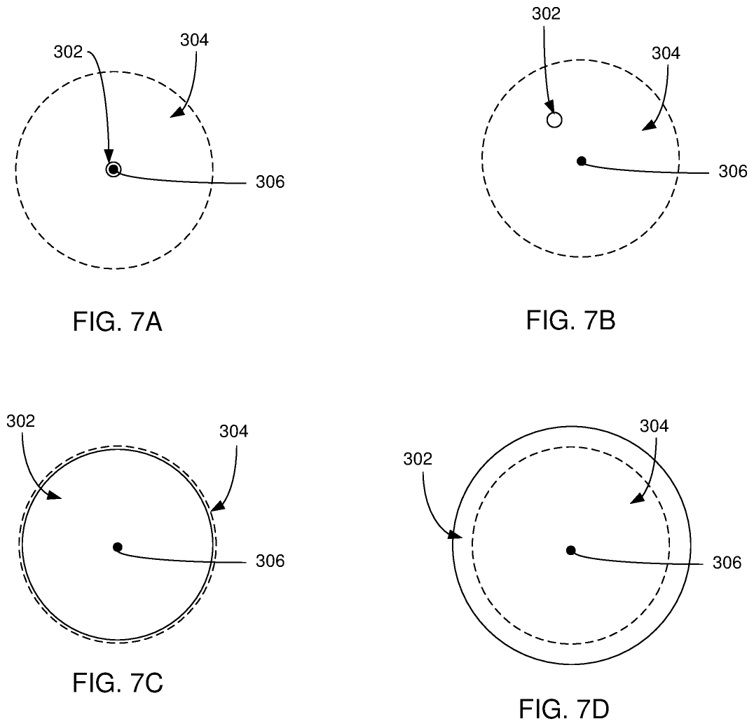

The temperature sensor 144 is aimed in nominally the same direction as the emission of light from the lighthead 110. In some embodiments, the arrangement of the temperature sensor is fixed relative to the lighthead. In the fixed arrangement, the temperature sensor measures the temperature of a predetermined location relative to the region of interest illuminated by the one or more light sources. For example, the predetermined location may correspond with the focal point of the lighthead. In many cases, this would be the temperature of the surgical site. In some examples, the predetermined location is a portion of the region of interest illuminated by the one or more light sources. FIG. 7A shows an example in which the predetermined location is defined by a region 302 that is within and smaller than the region of interest 304 and corresponds to the focal point 306 of the light emitted from the lighthead. FIG. 7B shows an example in which the predetermined location is defined by a region 302 that is within and smaller than the region of interest 304 and is different than the focal point 306 of the light emitted from the lighthead. In other examples, the predetermined location is defined by a region 302 that coincides with (FIG. 7C) or is larger than (FIG. 7D) the region of interest 304 illuminated by the one or more light sources. For example, the field of view of the camera may coincide with or may be larger than the region of interest. For purposes of determining temperature of the target within the region of interest, information from the video signal used in determining temperature may be limited to a defined region which may be defined and adjusted based on software or a graphical user interface.

In other embodiments, the alignment/arrangement of the temperature sensor 144 is adjustable such that the location within the region of interest illuminated by the one or more light sources can be adjusted. In some examples, the alignment of temperature sensor can be adjusted using a motor assembly 146 (FIG. 2) and may be controlled using one or more buttons on the handle or an interface external to the lighthead. In some embodiments, a light source 148 such as a laser may be aligned with the temperature sensor to emit a light beam to confirm alignment of the temperature sensor relative to the object. In embodiments in which the temperature sensor is embodied as a camera, image data from the camera can be used to visually confirm the alignment of the temperature sensor relative to the region of interest. For example, the camera may be paned and/or zoomed to adjust the field of view. The camera may be adjusted using one or more buttons on the handle or an interface external to the lighthead.

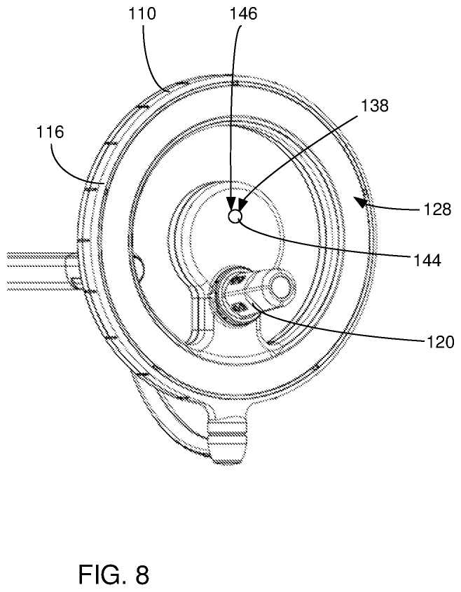

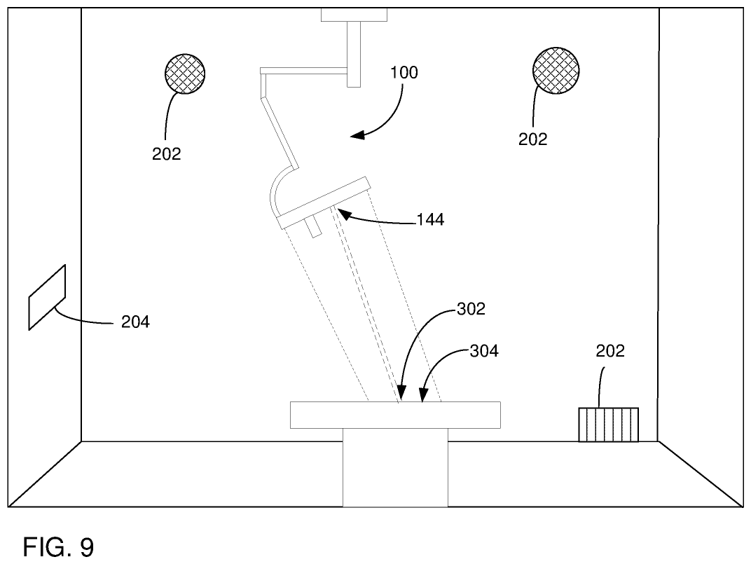

While the above-described embodiments include the temperature sensor housed within the handle of the lighthead, in other embodiments, the temperature sensor is included at a different location of the lighthead. For example, the temperature sensor may be attached to and/or disposed in the housing and arranged such that it is aimed in nominally the same direction as the emission of light from the lighthead. For example, FIGS. 8 and 9 shows an embodiment in which the temperature sensor 144 is mounted to the housing at the inner round portion 124 of the lighthead. In other examples, the temperature sensor is mounted to the housing at the annular shape outer portion 122 or the radially protruding arm 126. In embodiments in which there are multiple temperature sensors, the temperature sensor mounted to the housing at the annular shape outer portion 122, inner round portion 124, or radially protruding arm 126 can be used in combination with the sensor in the handle.

The temperature sensor may be any one of the temperature sensors described above with respect to the embodiments of FIGS. 1-7, and the alignment/arrangement/adjustment of the temperature sensor 144 relative to the region of interest may be the same as described in connection with FIGS. 7A-7D. The details thereof will not be repeated for sake of brevity.

It will be appreciated that in other embodiments, the temperature sensor can be located in a different location, external to the lighthead. So long as the location of the temperature sensor can detect the temperature at the surgical site, similar advantages can be provided.

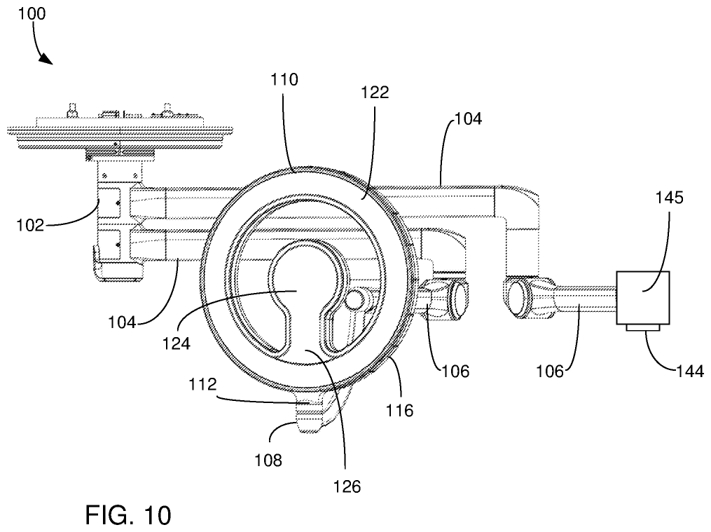

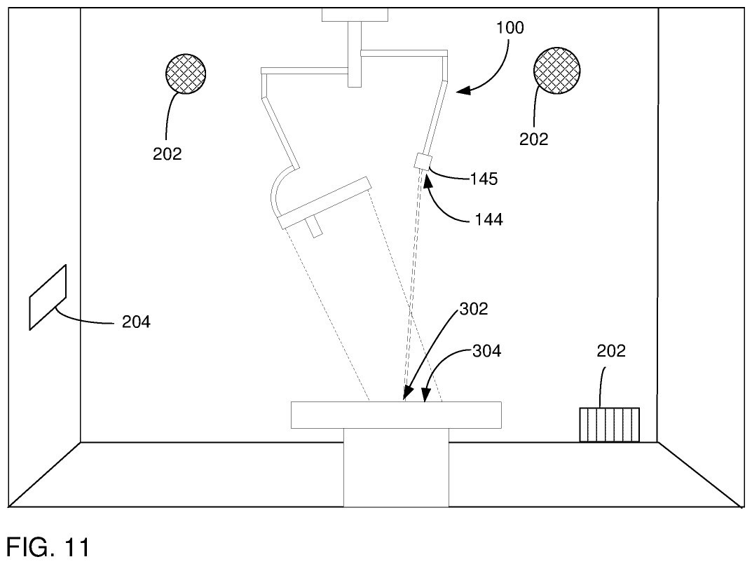

For example, FIGS. 10 and 11 show an example in which the medical device support system includes a designated suspension arm including a temperature sensor assembly 145 including a temperature sensor 144. The location of the temperature sensor assembly may be adjusted via manipulation of the suspension arm so that the temperature sensor is arranged to detect the temperature of the target in the region of interest.

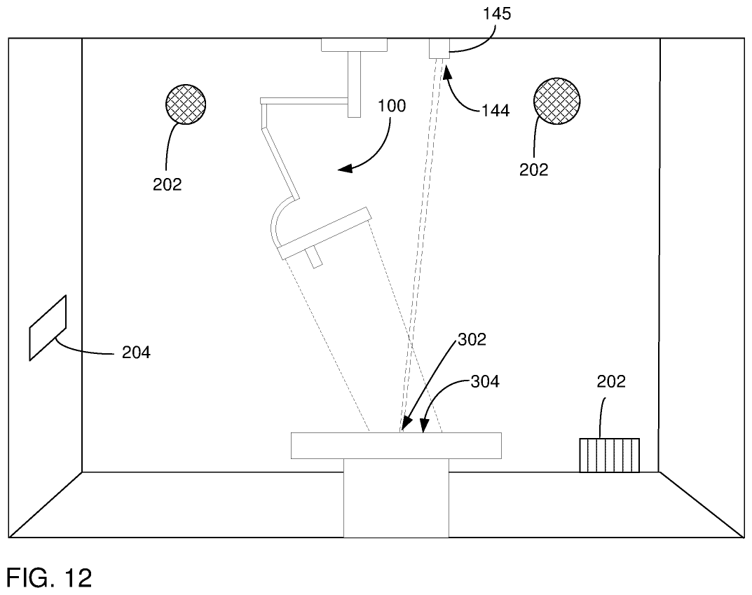

FIG. 12 shows another example in which a temperature sensor assembly 145 including a temperature sensor 144 is located at the ceiling. In some embodiments, a temperature sensor assembly 145 is attached to the ceiling. In other embodiments, the temperature sensor assembly 145 is integrated into a ceiling system. In still other embodiments (not shown) the temperature sensor assembly 145 is included on a designated boom system separate from the medical device support system and including a temperature sensor assembly. The location of the temperature sensor assembly may be adjusted via manipulation of the suspension arm so that the temperature sensor is arranged to detect the temperature of the target in the region of interest.

The temperature sensor in any of the embodiments described in FIGS. 10-12 may be any one of the temperature sensors described above with respect to the embodiments of FIGS. 1-7, and the alignment/arrangement/adjustment of the temperature sensor 144 relative to the region of interest may be the same as described in connection with FIGS. 7A-7D. The details thereof will not be repeated for sake of brevity.

It will be appreciated that in some embodiments, more than one temperature sensor may be included to measure the temperature of the location in the region of interest. For example, a system may include two or more temperature sensors, the arrangement/configuration of which being any combination of two or more of the above-described sensors.

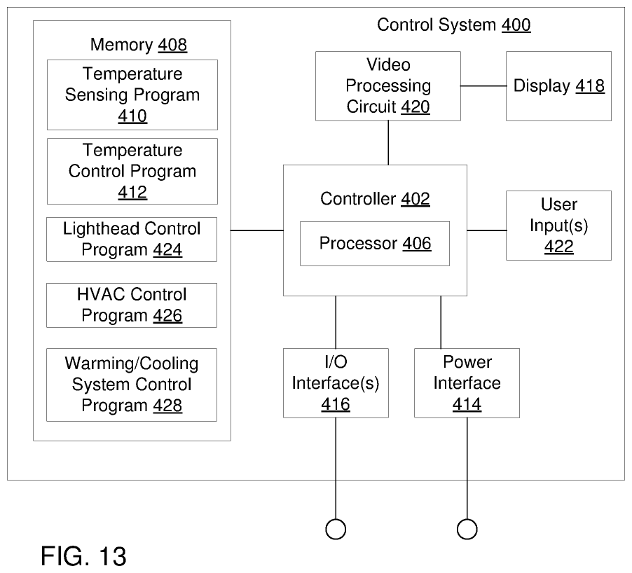

Referring now to FIG. 13, the temperature sensor is connected to a controller 402. The controller may be implemented as part of a control system 400 and may be configured to process an input from the temperature sensor 144 and control or provide an output for controlling temperature at the region of interest based at least in part on the temperature measured by the sensor 144. Exemplary control can include one or more of the control of light intensity and/or light output distribution from the lighthead, the control of one or more devices of an active warming/cooling system, and the control of one or more environmental temperature control systems.

In some embodiments, the control system 400 is provided in the handle or the lighthead. In other embodiments, the control system 400 is located external to the handle and lighthead, or even outside of the medical device support system 100. In other embodiments, the control system 400 is located in a combination of two or more of the handle 120, the lighthead housing 116, outside of the lighthead housing 116, and outside of the medical device support system 100.

The controller 402 is configured to carry out overall control of the functions and operations of the control system 400. The controller 402 may include a processor 406, such as a central processing unit (CPU), microcontroller, or microprocessor. The processor 406 executes code stored in a memory (not shown) within the controller 402 and/or in a separate memory, such as the memory 408, in order to carry out operation of the lighthead and temperature sensor. For example, the memory may contain stored data pertaining to the operation of the temperature sensor, and the processing of the signal received from the temperature sensor. The memory may contain stored data pertaining to the control of the lighthead, the control of one or more devices of an active warming/cooling system, and/or the control of one or more environmental temperature control systems based on the processed signal.

FIG. 13 shows an example in which a temperature sensing program 410 and a temperature control program 412 are stored in the memory 408. The temperature sensing program 410 and a temperature control program 412 may each be embodied in the form of executable logic routines (e.g., lines of code, software programs, etc.) that are stored on a non-transitory computer readable medium (e.g., the memory 408) and executed by the controller 402 (e.g., using the processor 406).

The memory 408 may be, for example, one or more of a buffer, a flash memory, a hard drive, a removable media, a volatile memory, a non-volatile memory, a random access memory (RAM), or other suitable device. In a typical arrangement, the memory 408 may include a non-volatile memory for long term data storage and a volatile memory that functions as system memory for the controller 402. The memory 408 may exchange data with the controller 402 over a data bus. Accompanying control lines and an address bus between the memory 408 and the controller 402 also may be present. The memory 408 is considered a non-transitory computer readable medium.

Operating power may be received from an external power source via a power interface 414.

The control system 400 may further include one or more input/output (I/O) interface(s) 416. The I/O interface(s) 416 may be in the form of one or more electrical connectors and may connect the controller 402 to one or more of the temperature sensor, light sources 118, lens control motor, one or more devices of an active warming/cooling system, one or more environmental temperature control systems, and/or one or more other controllers associated with the lighthead, the one or more devices of an active warming/cooling system, and/or the one or more environmental temperature control systems.

For example, the controller 402 may be communicatively coupled to the temperature sensor 144. The controller 402 may control operation of the temperature sensor, as well as the adjustment of the temperature sensor (alignment, focus, zoom, pan, etc.) The controller may also receive and process a signal from the temperature sensor 144. For example, the controller can process the electrical signal representative of radiant power to a temperature value. In another example, images or video can be processed to obtain a temperature value. The processing of the signal can be accomplished by the controller executing the temperature sensing program 410 using the signal as an input.

The control system 400 may include a display 418. In some embodiments, the display 418 can display video and/or images received from the temperature sensor 144. In some embodiments, the display 418 can display information such as temperature, set points, warnings, etc. The display may be a lighted display. In some embodiments, the display 418 is a backlit liquid-crystal display (LCD). In other embodiments, the display 418 is an organic light-emitting diode (OLED) display. The display 418 may be coupled to the controller 402 by a video processing circuit 420 that converts image and/or video data to an image and/or video signal used to drive the display 418. The video processing circuit 420 may include any appropriate buffers, decoders, video data processors and so forth.

The control system 400 may include one or more user inputs 422 for receiving user input for controlling operation of the control system 400. Exemplary user inputs 422 include, but are not limited to, a touch input that overlays the display 418 for touch screen functionality, one or more buttons such as those included on the handle or in a different location, and so forth.

The controller 402 may be communicatively coupled to one or more other components of the lighthead 110. In some embodiments, the controller 402 is communicatively coupled to the light emitting elements 118. In other embodiments, the controller 402 is communicatively coupled to the one or more motors configured to adjust the lens of the lighthead. The controller may control the intensity of the light at the region of interest based on the temperature value. This control can be accomplished by the controller executing the temperature control program 410 using the temperature value as an input. In some embodiments, this control can be accomplished by also executing a lighthead control program 424 stored in the memory 408 for controlling overall operation of the lighthead 110. In an example, the controller controls the radiant power of the light sources 118. In another example, the controller controls the position of the lens to adjust the light output distribution of the light sources.

Alternatively, the controller 402 may be communicatively coupled to a separate controller for controlling the one or more other components of the lighthead. The separate controller may be configured to carry out overall control of the one or more other components of the light head, and the separate controller may include a processor (e.g., CPU) for executing code stored in a memory. The one or more components of the lighthead may be controlled by the controller based at least in part on the value/signal received from controller 402.

In some embodiments, the controller 402 may be coupled to one or more environmental temperature control systems. For example, the controller 402 may be communicatively coupled to and may control a heating, ventilation and air-conditioning (HVAC) system. The controller 402 may control the operation of the HVAC system based at least in part on the temperature value. This control can be accomplished by the controller 402 executing the temperature control program 412 using the temperature value as an input. In some embodiments, this control can be accomplished by also executing a HVAC control program 426 stored in the memory 408 for controlling overall operation of the HVAC system. In another example, the controller 402 may be communicatively coupled to and may control the operation of one or more devices of an active warming/cooling system. This control can be accomplished by the controller 402 executing the temperature control program 412 using the temperature value as an input. In some embodiments, this control can be accomplished by also executing a warming/cooling system control program 428 stored in the memory 408 for controlling overall operation of the warming/cooling system.

Alternatively, the controller 402 may be communicatively coupled to one or more external control systems for controlling one or more environmental temperature control systems. The one or more external control systems may include, for example, a controller configured to carry out overall control of the functions and operations of the environmental temperature control system. The controller may include a processor (e.g., CPU) for executing code stored in a memory. The environmental temperature control system may be controlled by the controller based at least in part on the value received from controller 402. For example, the controller may be communicatively coupled to a controller for an HVAC system. The controller 402 can output the temperature value to the external controller, which in-turn can use the value to control the HVAC system. In another example, the controller may be communicatively coupled to a controller for an active warming/cooling system. The controller 402 can output the temperature value to the external controller, which can in-turn use the value/signal to control the active warming/cooling system.

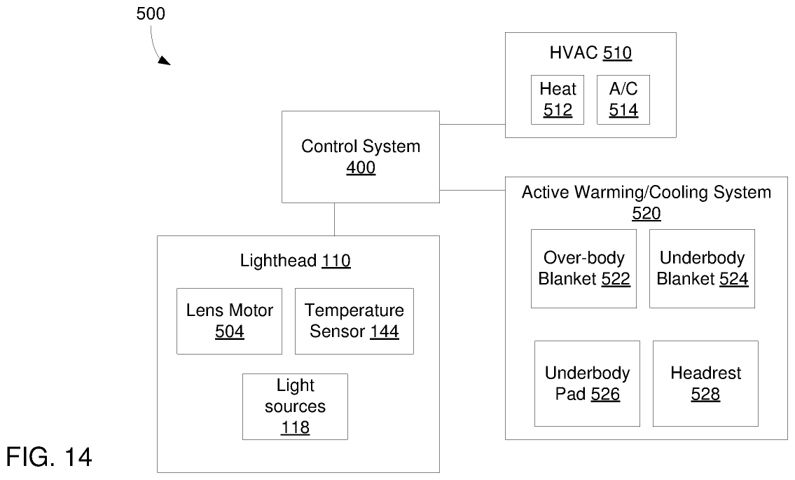

FIG. 14 shows an exemplary system. In the example shown, the control system 400 is coupled to the lighthead 110. The control system 400 is also coupled to an HVAC system 510 and an active warming/cooling system 520. While the control system 400 is schematically shown as being a separate unit from the lighthead 110, HVAC system 510, and active warming/cooling system 520, in other embodiments the control system 400 may be integrated within one or a combination of these systems.

In the example shown, the control system 400 can receive a signal from the temperature sensor 144 and can control one or both of the light sources 118 and the lens motor 504 in response to the received signal. For example, the control system 400 may increase or decrease the intensity of the light sources 118 and/or may narrow or widen the light output distribution from the lighthead by adjustment of the lens. The control system can also control the activation/deactivation and/or setpoints of the heat 512 and A/C 514 systems of the HVAC system 510. The control system 400 can also respectively control the activation/deactivation and/or setpoints of the devices associated with the active warming/cooling system 520. Exemplary warming or cooling peripheral devices include a warming or cooling over-body blanket 522, a warming or cooling underbody blanket 524, a warming or cooling underbody pad 526, and a warming or cooling headrest 528.

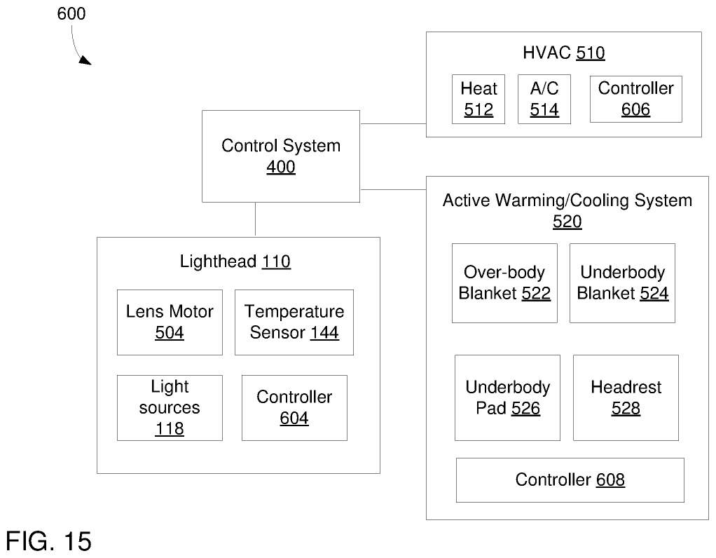

FIG. 15 shows another exemplary system. The system differs from that shown in FIG. 14 in that, in addition to the control system 400, additional controllers 604, 606, 608 are respectively associated with the lighthead, HVAC unit, and active warming/cooling system.

In the example shown, the control system 400 can receive a signal from the temperature sensor 144, process the signal, and can output one or more processed signals/values that are used as an input to the controller 604 of the lighthead, the controller 606 of the HVAC unit, and/or the controller 608 of the active warming/cooling system to control the temperature at the region of interest. For example, output of the processed signal from the control system 400 to the controller 604 of the lighthead may cause the controller of the lighthead to increase or decrease the intensity of the light sources and/or may narrow or widen the light output distribution from the lighthead. Output of the processed signal from the control system 400 to the controller 606 of the HVAC system may cause the controller 606 of the HVAC system to control the activation/deactivation of the heating system and A/C system of the HVAC unit. Output of the processed signal from the control system 400 to the controller 608 of the active warming/cooling system may cause the controller 608 of the active warming/cooling system to control the activation/deactivation and/or setpoints of the devices associated with the active warming/cooling system, such as an over-body blanket, underbody blanket, underbody pad, and headrest.

It will be appreciated that in other embodiments, the controllers of the system can be differently arranged and configured. For example, in some embodiments, the controller for the lighthead can be implemented similar to the controller 402 and control system 400 shown in FIG. 14 in that the controller can receive a signal from the temperature sensor and can control one or both of the light sources and the lens motor in response to the received signal. Additional controllers can respectively be provided the HVAC unit and active warming/cooling system similar to that shown in FIG. 15, and the control system of the lighthead can output one or more processed signals that are used as an input to the controller of the HVAC unit and/or the controller of the active warming/cooling system to control the temperature at the region of interest.

While FIGS. 14 and 15 show exemplary embodiments of systems including one lighthead, it will be appreciated that in other embodiments more than one lighthead is included in the system. One such exemplary embodiment is shown in FIG. 1. In such embodiments, the lightheads can be controlled collectively or individually. Each lighthead can include its own temperature sensor, a portion of the lightheads can include a temperature sensor, or only one of the lightheads can include a temperature sensor. In embodiments in which more than one temperature sensor is included, the temperature values can be averaged, or comparison to a temperature setpoint or range can be performed for each temperature value.

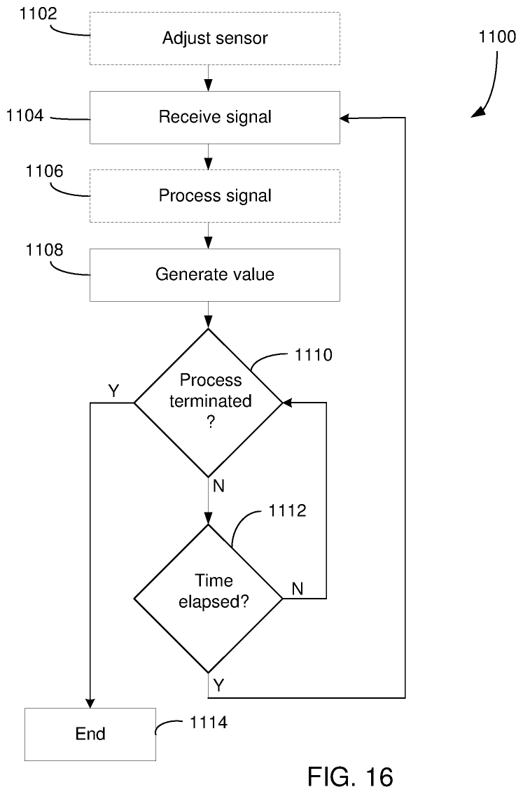

FIG. 16 is a flowchart showing an exemplary process 1100 for determining a measured temperature in the region of interest. In some embodiments, the process described in FIG. 16 is performed by the controller 402 executing the temperature sensing program 410. In embodiment in which more than one temperature sensor is included, the process 1100 may be performed for each sensor.

Optionally, at step 1102, the temperature sensor is adjusted. This may include alignment with at least a portion of the region of interest, zooming, panning, focusing, and the like. In some embodiments, the temperature sensor is already adjusted, or the temperature sensor is in a fixed arrangement relative to the region of interest, and therefore this step is omitted.

At step 1104, the signal is received from the temperature sensor. In some embodiments, the signal is an electrical signal indicative of the radiant power of the object in the region of interest. In other embodiments, the signal is image(s) and/or video of a field of view including at least a portion of the region of interest.

Optionally, at step 1106, the received signal is processed. For example, image(s) and/or video of the field of view can be selected and/or cropped for temperature determination. The portion of the image(s) and/or video that is used in determining the temperature can be converted to one or more values. The values can be averaged or weighted.

At step 1108 the temperature value is generated. In some embodiments, the temperature value is generated based on the electrical signal indicative of the radiant power of the object in the region of interest. In some embodiments, the temperature value is generated based on the one or more values from the image(s) and/or video.

At step 1110, it is determined whether the process is terminated. If yes, the process ends at step 1114. If no, the process proceeds to step 1112 where it is determined whether a predetermined time has lapsed.

If the predetermined time has not elapsed (no), the process reverts to step 1110. If the predetermined time has elapsed (yes), the process returns to step 1104.

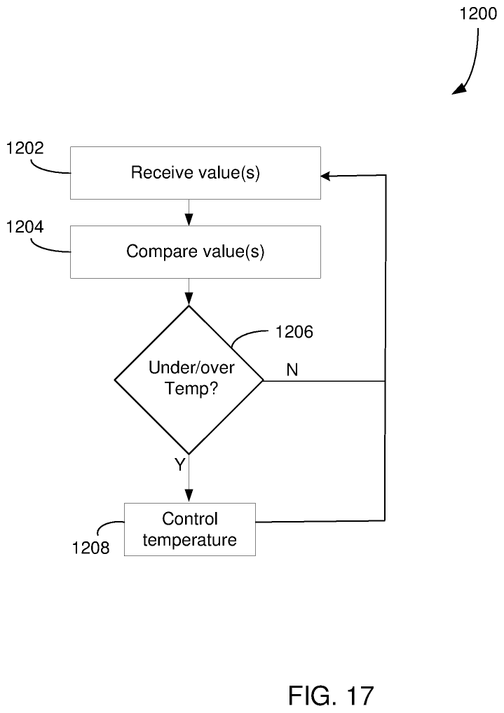

FIG. 17 is a flowchart showing an exemplary process 1200 for controlling temperature at the region of interest based at least in part on the temperature measured by the sensor. In some embodiments, the process described in FIG. 17 is performed by the controller 402 executing the temperature control program 412.

At step 1202, the temperature value is received. In some embodiments, a single temperature value is received. In other embodiments, such as those in which more than one temperature sensor is present, two or more values are received.

At step 1204, the received value(s) is compared against a set point or set temperature range. In some embodiments, the received value is compared as an individual number. In embodiments in which more than one temperature sensor is included as a part of the system, each received value may be individually compared. In other embodiments, the received value is compared as part of a group of two or more numbers. For example, the received value can be compared as part of a rolling average of temperature values over a predetermined time period. For example, an average of the temperature values received over the past minute may be used. This can be performed for the temperature received from each temperature sensor. In another example in which there is more than one temperature sensor, an average of the values from the temperature sensors for a given point in time can be used for the comparison.

At step 1206, it is determined whether the received temperature(s) is outside of the setpoint or the predetermined range. If no, the process reverts back to step 1202. If yes, the process proceeds to step 1208 to control the temperature at the region of interest. In some embodiments, the intensity of the light provided to the surgical site is adjusted. For example, the lighting elements 118 are adjusted and/or the light output distribution is adjusted. In other embodiments, the environment temperature (room temperature) is adjusted. In other embodiments, the temperature of one or more active warming/cooling devices is adjusted. Control may also result in instruction for an individual to adjust/manipulate one or more blankets, drapes, saline bottles, or other passive items proximate the region of interest. The process may then revert to step 1202.

The control results in adjustment of the temperature at the region of interest to the setpoint or to be within the predetermined temperature range. In a situation where the temperature is above the predetermined range, the control can cause the temperature at the site to lower. In a situation where the temperature is below the predetermined range, the control can cause the temperature at the site to rise.

It will be appreciated that in some embodiments, the process 1200 may include a time delay from when the received value(s) is determined to be over the setpoint or the predetermined range (step 1206) until when the temperature at the region of interest is controlled (step 1208). For example, upon determining at step 1206 that the temperature is over or under (step 1206, yes), it may be subsequently determined whether a predetermined amount of time has elapsed. This predetermined amount of time may be any suitable amount of time (e.g., 10 seconds, 30 seconds, 1 minute, etc.) and may be compared to an amount of time that the instance of the over condition has been occurring. For example, when consecutive over values are read, the total amount of time over which the consecutive readings are read may be compared to the predetermined amount of time. If the total amount of time is less than the predetermined amount of time, the process may revert to step 1202 without adjusting the light output. If the total amount of time is equal to or greater than the predetermined amount of time, the process may proceed to step 1208 to control temperature. The total amount of time may reset once the received value(s) is no longer over. This approach may allow for minor incidental fluctuations in the environmental conditions.

Although the invention has been shown and described with respect to certain preferred embodiments, it is understood that equivalents and modifications will occur to others skilled in the art upon the reading and understanding of the specification and the attached drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application. The present invention includes all such equivalents and modifications and is limited only by the scope of the following claims.

1. A system, comprising:
a surgical lighthead including a lighthead housing including one or more light emitting elements therein that are arranged to emit light toward a region of interest;
a temperature sensor mounted to the lighthead housing and arranged such that it measures the temperature of a location in the region of interest; and
a controller operatively coupled to the temperature sensor and configured to control temperature at the region of interest based at least in part on the temperature measured by the temperature sensor.

2. The system of claim 1, wherein the temperature sensor is an infrared thermometer.

3. The system of claim 1, wherein the temperature sensor is an infrared camera.

4. The system of claim 1, further comprising a handle mounted to the lighthead housing, wherein the temperature sensor is within the handle.

5. The system of claim 1, wherein the region of interest is a surgical site.

6. The system of claim 1, wherein the controller is configured to control environmental temperature based at least in part on the temperature measured by the temperature sensor.

7. The system of claim 1, wherein the controller is configured to control an HVAC system based at least in part on the temperature measured by the temperature sensor.

8. The system of claim 1, wherein the controller is configured to control a warming or cooling blanket based at least in part on the temperature measured by the temperature sensor.

9. The system of claim 1, wherein the controller is configured to control a warming or cooling underbody pad based at least in part on the temperature measured by the temperature sensor.

10. The system of claim 1, wherein the controller is configured to control a warming or cooling peripheral device based at least in part on the temperature measured by the temperature sensor.

11. The system of claim 1, wherein the controller is configured to control intensity of the one or more light emitting elements based at least in part on the temperature measured by the temperature sensor.

12. The system of claim 1, wherein the controller is configured to control light output distribution of the one or more light emitting elements based at least in part on the temperature measured by the temperature sensor.

13. A surgical lighting system, comprising:
a lighthead housing including one or more light emitting elements therein that are arranged to emit light toward to a surgical site;
a handle mounted to the lighthead housing, the handle including a handle housing having a sufficient size to be gripped by the human hand; and
a temperature sensor mounted within the handle housing and arranged such that it measures the temperature of a location in the surgical site, wherein the temperature sensor is one of an infrared thermometer or an infrared camera.

14. (canceled)

15. (canceled)

16. A system, comprising:
a medical device suspension system;
a surgical lighthead mounted to the medical device suspension system and comprising a lighthead housing including one or more light emitting elements therein that are arranged to emit light toward to a surgical site;
a temperature sensor external to the lighthead and configured to measure the temperature of a location in the surgical site; and
a controller operatively coupled to the temperature sensor and configured to control temperature at the region of interest based at least in part on the temperature measured by the temperature sensor.

17. The system of claim 16, wherein the temperature sensor is one of an infrared thermometer or an infrared camera.

18. (canceled)

19. The system of claim 16, wherein the temperature sensor is mounted to the medical device suspension system.

20. The system of claim 16, wherein the temperature sensor is not mounted to the medical device suspension system.

21. (canceled)

22. The system of claim 16, wherein the controller is configured to control room temperature based at least in part on the temperature measured by the temperature sensor.

23. The system of claim 16, wherein the controller is configured to control a patient warming or cooling system based at least in part on the temperature measured by the temperature sensor.

24. The system of claim 16, wherein the controller is configured to control a warming or cooling blanket based or a warming or cooling underbody pad at least in part on the temperature measured by the temperature sensor.

25. (canceled)

26. The system of claim 16, wherein the controller is configured to control a warming or cooling peripheral device based at least in part on the temperature measured by the temperature sensor.

27. The system of claim 16, wherein the controller is configured to control intensity or the light output distribution of the one or more light emitting elements based at least in part on the temperature measured by the temperature sensor.

28. (canceled)
